(19) United States
(12) Patent Application Publication
(10) Pub. No.: US 2003/0031734 A1
(43) Pub. Date: Feb. 13, 2003

(54) Extracts of blueberries with anti-oxidant and anti-cancer properties

(76) Inventor: Robert T. Rosen, Monroe Township, NJ (US)

Correspondence Address: Licata & Tyrrell P.C., 66 E. Main Street, Marlton, NJ, 08053, US

(21) Appl. No.: 10/175,216

(22) Filed: Jun. 17, 2002

Related U.S. Application Data
(60) Provisional application No. 60/311,503, filed on Aug. 13, 2001.

(51) Int. Cl. A61K 35/78

(52) U.S. Cl. 424/732; 424/777

(57) ABSTRACT

Compositions and methods for treating and preventing cancer are provided based on administration of a highbush blueberry extract.

### INTRODUCTION

This application claims the benefit of priority from U.S. provisional application Serial No. 60/311,503, filed Aug. 13, 2001.

### BACKGROUND OF THE INVENTION

Naturally occurring non-nutritive agents present in plants are believed to have disease preventive properties. It is well-known that consumption of adequate amounts of fruits and vegetables is associated with a lowered risk of degenerative diseases such as cancer (Ames, B. N. et al. 1995. Proc. Natl. Acad. Sci. USA 92:5258-5265). Extensive research has begun into identifying particular plants and plant extracts that have disease prevention or curative properties, including plants or plant extracts that have anti-cancer activity.

Blueberries are a fruit that is produced commercially in North America, Europe and Japan. It is consumed fresh or as a processed product. The chemical components of blueberry have not been extensively studies, although anthocyanins are known to be a major chemical component of the fruit. The major anthocyanins identified in highbush blueberry include malvidin 3-galactoside, delphinidin 3-galactoside, delphinidin 3-arabinoside, petunidin 3-galactoside, petunidin 3-arabinoside, and malvidin 3-arabinoside (Gao, L. and G. Mazza. 1994. J. Food Sci. 59:1057-1059). There are reports of flavonols and hydroxybenzoic acid derivatives in blueberry (Gao, L. and G. Mazza. 1994. J. Food Sci. 59:1057-1059; Bilyk, A. and G. M. Sapers. 1986. J. Agric. Food Chem. 34:585-588; Piironen, V. et al. 1986. J. Agric. Food Chem. 34:742-745; Teeling, C. G. V. et al. 1971. J. Food Sci. 36:1061-1063; Schuster, B and K. Hermann. 1985. Phytochem. 24:2761-2764).

Extracts of the fruits of Vaccinium species, to which the blueberry belongs, have been shown to have anti-cancer activity in vitro (Bomser, J. et al. 1996. Planta Med. 62:212-216). Specific fractions of the crude extract were shown to have the potential to inhibit the initiation and promotion phases of chemical carcinogenesis. The potential anti-carcinogenic effects were suggested to be attributed to one of a variety of compounds in the crude extract including lipids, sterols, carotenoid, chlorophyll, or proanthocyanin.

The natural anti-oxidant capacity of certain foods has been linked to anti-cancer activity. Blueberry was rated as highest in anti-oxidant content among over 40 fruits and vegetables examined (Prior, R. L. and G. Cao. 1998. J. Agri. Food Chem. 46:2686-2693). In general, anti-oxidant capacity of plants has been correlated with high content of phenolic and anthocyanin compounds. In a study of the anti-oxidant activity and total phenolic content of selected fruits, vegetables, and grain products, blueberry was shown to have the highest level of anti-oxidant activity (Velioglu, Y. S. and G. Mazza. 1998. J. Agri. Food Chem. 46:4113-4117). Blueberry had twice the anti-oxidant capacity of either raspberry or strawberry and the anti-oxidant capacity did not change during storage (Kalt, W. et al. 1999. J. Agri. Food Chem. 47:4638-4644). In a feeding study in rats, strawberry, spinach and blueberry fed to 19-month old Fischer 344 rats for 8 weeks as dietary supplements were shown to be effective in reversing age-related deficits in several neuronal and behavioral parameters (Joseph, J. A. and B. Shukitt-Hale. 1999. J. Neuroscience 19:8114-8121). These data suggested that phytochemicals present in blueberry as well as other phytochemicals in anti-oxidant rich foods may be beneficial in reversing the course of aging.

Studies have been performed to examine the types of compounds found in blueberry. The major classes of compounds currently identified include anthocyanins, proanthocyanidins, and hydroxycinnamic acid compounds and their derivatives.

It has now been found that an extract of blueberries has biological activity as both an anti-oxidant and a cytotoxic agent. Such an extract would be useful as an anti-cancer agent.

### SUMMARY OF THE INVENTION

An object of the present invention is an extract of highbush blueberry comprising urosolic acid, 3β,19α-dihydroxy-urs-12-en-28-oic acid, gallic acid and procatechuric acid. In another embodiment the extract is an anti-oxidant extract and comprises gallic acid and procatechuric acid. In yet another embodiment the extract is a tumor cell growth inhibitor and comprises urosolic acid and 3β,19α-dihydroxy-urs-12-en-28-oic acid.

Another object of the present invention is a method for inhibiting tumor cell growth in an animal comprising administering to an animal the extracts of the present invention.

Yet another object of the present invention is a method for inhibiting oxidative activity in an animal comprising administering to an animal the anti-oxidant extract of the present invention.

Another object of the present invention is a method for preventing or treating cancer in an animal comprising administering to an animal an effective amount of an extract of the present invention.

### DETAILED DESCRIPTION OF THE INVENTION

Compositions are provided which comprise extracts of blueberry. Specifically, the phenolic acid compounds in an ethyl acetate fraction of highbush blueberry have been shown to possess both anti-oxidant and anti-carcinogenic activity. Therefore, these compositions provide a method for prevention and treatment of cancer in animals, including humans.

Experiments were performed to isolate and identify components in dried fruits of blueberry. The dried fruits were first extracted with 95% ethanol at room temperature.

The resulting extract was evaporated to dryness, the residue dissolved in water, and the aqueous solution extracted with hexane (H), ethyl acetate (E), and n-butanol (B) to give three fractions of 25 g, 17.3 g and 150 g, respectively.

The E fraction was subjected to column chromatography on silica gel, eluted with hexane:ethyl acetate (10:1, 9:1, 8:1, 7:1, 6:1, 5:1, 4:1, 3:1, 2:1, 1:1, each 3500 ml) and separated into 15 fractions as determined by thin layer chromatography. Fraction 7 was then rechromatographed on Sephadex LH-20, eluted with methanol, and purified with column chromatography (chloroform:methanol, 40:1) to give 180 mg of compound 1. Fraction 8 was rechromatographed on Sephadex LH-20, eluted with methanol, and then subjected to column chromatography on silica gel (hexane:ethyl acetate, 3:1) to yield 24 mg of compound 2. Fraction 9 was rechromatographed on silica column chromatography, eluted with chloroform:ethyl acetate (10:1), chloroform:ethyl ether (2:1), and chloroform:methanol (30:1) to yield 350 mg of compound 3. Fraction 13 was first subjected to column chromatography on silica gel (chloroform:methanol:acetic acid, 20:1:1) and then purified by Sephadex LH-20 column to yield 70 mg of compound 4. Fraction 14 was first rechromatographed on Sephadex LH-20 column, then applied to silica gel columns, eluted with hexane:acetone:acetic acid (2:1:0.1), hexane:ethyl acetate: acetic acid (2:1:0.1), and chloroform:methanol:acetic acid (20:1:0.5) to yield 40 mg of compound 5 and 50 mg of compound 6. Fraction 15 was eluted with acetone:hexane:acetic acid (1:2:0.1) and chloroform:acetone:acetic acid (5:1:0.1), respectively, to yield 50 mg of compound 7.

The structure and identification of the seven isolated compounds was then determined using NMR. Compounds 1 through 7 were identified as: Compound 1: urosolic acid; compound 2: 3β,19α-dihydroxy-urs-12-en-28-oic acid; compound 3: 5-hydroxymethyl-2-furfural; compound 4: sitosterol-β-D-glucoside; compound 5: syringic acid; compound 6: gallic acid; and compound 7: procatechuric acid. Verification after NMR was made by comparing mass spectra with the literature and by comparing the spectra with authentic compounds using thin layer chromatography.

Once the compounds had been identified, experiments were performed to characterize the pharmacological activity of each compound. Using a well-established screening assay for cytotoxicity, the compounds were tested in a human leukemia cell line (HL-60) for their ability to inhibit growth of these cells in culture. This assay is routinely used by those of skill to determine the potential anti-carcinogenic activity of chemicals where anti-carcinogenic activity is correlated with growth inhibitory activity in HL-60 cells. Uptake of tritiated thymidine into cells, a measure of the level of DNA synthesis and thus cell growth, was determined in the cells both with and without the presence of each of the seven blueberry extract compounds. Two compounds, urosolic acid and 3β,19α-dihydroxy-urs-12-en-28-oic acid, were shown to effectively inhibit DNA synthesis in HL-60 cells, and thus inhibited cell growth. The IC₅₀ values were 1.5 ppm for urosolic acid and 1 ppm for 3β,19α-dihydroxy-urs-12-en-28-oic acid. Urosolic acid was also shown to produce significant cytotoxicity in lymphocytic leukemia cells p388 and L-210 as well as in human lung carcinoma cells A-549. Urosolic acid has also been isolated from apples, pears and other fruits and has been shown to have marginal cytotoxicity in KB and human colon (HCT-8) and mammary (MCF-7) tumor cells (Lee, K. H. et al. 1988. Planta Med. 308-311).

The potential anti-oxidative properties of the seven compounds was also tested. Experiments were performed using the Rancimat method, a common method for measurement of antioxidant activity of both synthetic and natural anti-oxidants.

The method is based on measuring changes in electrical conductivity of water caused by the formation of short-chain compounds when fats and oils are oxidized under elevated temperature and accelerated aeration. Using lard samples, the effects of each of the seven compounds on the oxidative stability of lard was determined. The anti-oxidative activity levels of the compounds isolated from blueberry are shown below in Table 1. The levels of anti-oxidant activity can be compared with a known anti-oxidant compound BHT.

TABLE 1
Anti-oxidative Activity of Blueberry Extract Compounds Using
the Rancimat Test

| Compound | Induction Time (hr) | Anti-oxidant Index |
|---|---|---|
| BHT | 5.8 | 2.76 |
| (positive control) | | |
| Urosolic Acid | 2.2 | 1.05 |
| 3β,19α-Dihydroxy- | 2.2 | 1.05 |
| urs-12-en-28-oic | | |
| Acid | | |
| 5-Hydroxymethyl-2- | 2.1 | 1.00 |
| furfural | | |
| Sitosterol-β-D- | 2.4 | 1.14 |
| glucoside | | |
| Syringic Acid | 3.1 | 1.48 |
| Gallic Acid | 28.1 | 13.38 |
| Procatechuric Acid | 18.5 | 8.81 |
| Lard | 2.1 | 1.00 |
| (negative control) | | |

Two of the seven compounds tested, gallic acid and procatechuric acid exhibited high oxidation-inhibition activity. Phenolic compounds such as these two compounds are widely distributed in plants and many similar compounds have been shown to be active as anti-oxidants, with anti-oxidative activity being ascribed to the hydroxyl groups (Chen, J. H. and C. T. Ho. 1997. J. Agric. Food Chem. 45:2374-2378).

Taken together, these studies have demonstrated the potential anti-carcinogenic and anti-oxidative effects of blueberry extracts. The anti-carcinogenic activity has been identified in at least specific extract components, urosolic acid and 3β,19α-dihydroxy-urs-12-en-28-oic acid, while the anti-oxidative capacity has been linked to two other components of the blueberry extract, gallic acid and procatechuric acid.

Based upon the experiments described herein, it is believed that compositions comprising highbush blueberry extract including but not limited to urosolic acid, 3α,19α-dihydroxy-urs-12-en-28-oic acid, gallic acid and procatechuric acid, may be included in foods and dietary supplements or “nutraceuticals” for prevention or treatment of cancer. One of skill can use the results of experiments in cells described herein to determine effective amounts to be administered to animals, including humans. By “effective amount” it is meant a concentration that inhibits tumor growth (an anti-cancer effect) either in vitro in cells or in vivo in animals. For example, human test doses can be extrapolated from effective doses in cell studies, such as IC₅₀ values, or from effective doses in vivo by extrapolating on a body weight or surface area basis. Such extrapolations are routine in the art.

Compositions comprising highbush blueberry extracts can be formulated for administration as a food supplement using one or more fillers. Alternatively, compositions comprising these extracts can be administered as conventional pharmaceuticals using one or more physiologically acceptable carriers or excipients. Nutraceutical compositions can be formulated for administration by any route including, but not limited to, inhalation or insufflation (through mouth or nose), oral, buccal, parenteral, vaginal, or rectal administration. In one embodiment, oral administration, the compositions are added directly to foods and ingested as part of a normal meal. Various methods are known to those skilled in the art for addition or incorporation of nutraceuticals into foods.

Compositions for use in the present invention can also be administered in the form or tablets or capsules prepared by conventional means with pharmaceutically acceptable excipients such as binding agents, fillers, lubricants, disintegrants, or wetting agents. Examples of specific compounds for use in formulating tablets and capsules are described in detail in the U.S. Pharmacopeia. Tablets comprising the extract can also be coated by methods well known in the art. Liquid preparations for oral administration can also be used. Liquid preparations can be in the form of solutions, syrups or suspensions, or a dry product for reconstitution with water or another suitable vehicle before use. Such liquid preparations can be prepared by conventional means with pharmaceutically acceptable additives such as suspending agents, emulsifying agents, non-aqueous vehicles, and preservatives. Again, specific additives are well known to those of skill and are listed in places such as the U.S. Pharmacopeia. In one embodiment, the oral preparation is formulated to provide controlled time release of the active nutraceutical components. For buccal administration the extract can be formulated as a tablet or lozenge.

For administration by inhalation, compositions for use in the present invention can be delivered in the form of an aerosol spray in a pressurized package or as a nebulizer, with use of suitable propellants. In the case of a pressurized aerosol, the dosage unit can be determined by providing a valve to deliver a metered dose.

Parenterally administered compositions are formulated to allow for injection, either as a bolus or as a continuous infusion. Formulations for injection can be prepared in unit dosage forms, such as ampules, or in multi-dose units, with added preservatives. The compositions for injection can be in the form of suspensions, solutions, or emulsions, in either oily or aqueous vehicles. They may also contain formulatory agents such as suspending agents, stabilizing agents, and/or dispersing agents. The active ingredient may also be presented in powder form for reconstitution with a suitable vehicle before use. Specific examples of formulating agents for parenteral injection are found in the U.S. Pharmacopeia.

For rectal administration or vaginal administration, compositions for use in of the present invention can be formulated as suppositories, creams, gels, or retention enemas.

For dietary supplements, the extract can be added in concentrations up to 5% by weight and mixed according to methods routine in the art. Dietary supplements for animals can be prepared in a variety of forms including, but not limited to, liquid, powder, or solid pill forms. In the present invention, the highbush blueberry extract can administered either alone or in combination with other phytochemicals known to affect tumor cell growth, where combining compounds or extracts would lead to synergistic effects.

The following non-limiting example is presented to further illustrate the claimed invention:

### EXAMPLE

### Testing for Cytotoxicity in Human Leukemia Cells (HL-60)

HL-60 cells were cultivated in 1.62% RPMI 1640 medium supplemented with 10% fetal calf serum and 1% penicillin-streptomycin. The concentration of the cells in the medium was kept between 2 and 10×10⁵ counts/ml. Cell concentrations were counted at the beginning of each experiment with a microscope. Cells were washed without antibiotics and collected by centrifugation. The cells were then diluted to 3×10⁵ counts/ml with cold medium without antibiotics. Tritiated thymidine was mixed into the cold medium at a concentration of 150 Ci/mmol. The samples to be tested were dissolved in DMSO at concentrations between 0.1 g/L and 50 ug/L. A 2 microliter sample of each concentration was added to 6 ml tubes, using triplicate tubes for reproducibility. A 1 ml sample of the HL-60 cell culture was added to each tube, diluted 500-fold and mixed. The tubes were incubated at 37 C for 90 minutes and then cooled over ice. Cold PBS was added to each tube and they were centrifuged at 1000 rpm for 5 minutes. The supernatant was discarded and distilled water mixed with the cells. Free and bound tritiated thymidine levels were measured by scintillation counting.

What is claimed is:

1. An extract of highbush blueberry comprising urosolic acid, 3β,19α-dihydroxy-urs-12-en-28-oic acid, gallic acid and procatechuric acid.

2. An anti-oxidant extract of highbush blueberry comprising gallic acid and procatechuric acid.

3. A tumor cell growth inhibitor comprising an extract of highbush blueberry.

4. The tumor cell growth inhibitor of claim 3 wherein said extract comprises urosolic acid and 3β,19α-dihydroxy-urs-12-en-28-oic acid.

5. A method for inhibiting tumor cell growth in an animal comprising administering to an animal the extract of claim 1.

6. A method for inhibiting tumor growth in an animal comprising administering to an animal the inhibitor of claim 3.

7. A method for inhibiting oxidative activity in an animal comprising administering to an animal the extract of claim 1.

8. A method for inhibiting oxidative activity in an animal comprising administering to an animal the anti-oxidant extract of claim 2.

9. A method for preventing or treating cancer in an animal comprising administering to an animal an effective amount of an extract of claim 1.
